(19) United States
(12) Patent Application Publication
(10) Pub. No.: US 2015/0120205 A1
(43) Pub. Date: Apr. 30, 2015

(54) SYSTEM AND METHOD FOR MANAGING STRESS

(71) Applicant: Samsung Electronics Co., Ltd., Gyeonggi-do (KR)

(72) Inventors: Seung Young JEON, Gyeonggi-do (KR); Jeong Min Park, Gyeonggi-do (KR); Jae Yung Yeo, Gyeonggi-do (KR); Seung Eun Lee, Seoul (KR); Pyeong Gyu JIN, Gyeonggi-do (KR); Eun Jung Hyun, Seoul (KR)

(73) Assignee: Samsung Electronics Co., Ltd.

(21) Appl. No.: 14/485,233

(22) Filed: Sep. 12, 2014

(30) Foreign Application Priority Data
Oct. 24, 2013 (KR) 10-2013-0126869

(51) Int. Cl. A61B 5/16; A61B 5/01

(52) U.S. Cl. CPC A61B 5/165; A61B 5/015
USPC 702/19

(57) ABSTRACT

A device for managing user stress is provided. The device includes a collection unit that collects stress related information, an analysis unit that determines a stress state, and an expression unit provides a stress related expression to a user. The collection unit may receive feedback from the user, and the feedback may be used for determining the stress state.

### PRIORITY

The present application claims priority to Korean Patent Application No. 10-2013-0126869, which was filed in the Korean Intellectual Property Office on Oct. 24, 2013, the entire disclosure of which is incorporated herein by reference.

### BACKGROUND

1. Field of the Invention

The present invention relates generally to a method and apparatus for managing user stress based on information collected using a user terminal.

2. Background of the Invention

In general, people visit a medical institution to receive a diagnosis and receive treatment based on a diagnosis, when they feel pain in their body or believe that their condition has worsened. In addition, people often have a regular medical check-up at least once a year. In order to have these types of medical check-ups, people first need to make appointments with a medical institution such as a hospital and then visit the medical institution.

Additionally, people change their daily habits or take prescription drugs according to the results of examinations and advice and diagnoses related to the results.

However, these conventional methods involve severe temporal/spatial constraints and do not always verify a user's health condition. Also, these conventional methods are not suitable for analyzing a user's state of mental stress and providing information related thereto. Thus, a need exists for a method and apparatus to continuously verify/manage a user's health condition.

### SUMMARY

The present invention is designed to address at least the problems and/or disadvantages described above and to provide at least the advantages described below.

Accordingly, an aspect of the present invention is to continuously verify and manage a user's mental health condition by using a portable device.

Another aspect of the present invention is to minimize inconvenience resulting from typical stress analyzing methods requiring users to reply to too many questions and input too many items, by continuously verifying a user stress state without direct input by the user.

Another aspect of the present invention is to enhance the accuracy and reliability of stress determination, by continuously modifying a stress determining criterion suitable for users, based on feedback collected from the users.

In accordance with an aspect of the present invention, a device for managing user stress is provided. The device includes a collection unit that collects stress related information; an analysis unit that determines a stress state based on the collected information; and an expression unit that provides a stress related expression to the user, based on the determined stress state. The collection unit receives feedback on the provided stress related expression from the user, and the analysis unit uses the feedback to determine the stress state.

In accordance with another aspect of the present invention, a method is provided for managing stress of a user in a device of the user. The method includes collecting, by the device, stress related information; analyzing a stress state of the user, based on the collected information; providing a stress related expression to the user, based on the analyzed stress state; and receiving feedback from the user, based on the provided stress related expression. The feedback is used for analyzing the stress state.

In accordance with another aspect of the present invention, a method is provided for managing stress of a user in a device of the user. The method includes collecting, by the device, stress related information; transmitting the collected information to a server; receiving, from the server, an expression related to the stress; providing the received expression to the user; receiving feedback from the user, based on the provided expression; and transmitting the feedback to the server. The feedback is used by the server for analyzing the stress state of the user.

In accordance with another aspect of the present invention, a method is provided for managing stress of a user. The method includes collecting, by a first device, stress related information; analyzing a stress state, based on the collected information; and transmitting the analyzed stress state to a second device. The transmitted stress state is used for providing, to the user of the second device, an expression related to the stress of the user of the first device.

### BRIEF DESCRIPTION OF THE DRAWINGS

The above and other aspects, features, and advantages of certain embodiments of the present invention will be more apparent from the following detailed description taken in conjunction with the accompanying drawings, in which:

FIG. 1 illustrates a device for collecting stress related information according to an embodiment of the present invention;

FIG. 2 illustrates a system for storing stress related information according to an embodiment of the present invention;

FIG. 3 illustrates a device for providing stress related expressions according to an embodiment of the present invention;

FIG. 4 illustrates a device providing stress management according to an embodiment of the present invention;

FIG. 5 illustrates a distribution of stress values according to an embodiment of the present invention;

FIG. 6 illustrates an analysis unit and an expression unit of a device for providing stress management according to an embodiment of the present invention;

FIGS. 7A to 7E illustrate examples for expressing stress situations according to embodiments of the present invention;

FIGS. 8A and 8B illustrate examples for expressing stress states to a user according to embodiments of the present invention;

FIGS. 9A and 9B illustrate examples for expressing stress situations according to an embodiment of the present invention;

FIG. 10 illustrates a method for providing stress related information in linkage with externally received data according to an embodiment of the present invention;

FIG. 11 illustrates a method for a plurality of devices to cooperate and provide stress states to users according to an embodiment of the present invention;

FIG. 12 is a flow chart illustrating a method for managing user stress in a user device according to an embodiment of the present invention;

FIG. 13 is a flow chart illustrating a method for managing stress based on feedback received from a user according to an embodiment of the present invention;

FIG. 14 is a signal flow diagram illustrating a method for managing stress based on feedback received from a user according to an embodiment of the present invention;

FIG. 15 illustrates a method for managing user stress in a user device according to an embodiment of the present invention; and

FIG. 16 is a signal flow diagram illustrating a method in which a plurality of devices cooperate and manage user stress according to an embodiment of the present invention.

### DETAILED DESCRIPTION OF EMBODIMENTS OF THE INVENTION

Various embodiments of the present invention will now be described in detail with reference to the accompanying drawings. In the following description, specific details such as detailed configuration and components are merely provided to assist the overall understanding of these embodiments of the present invention. Therefore, it should be apparent to those skilled in the art that various changes and modifications of the embodiments described herein can be made without departing from the scope and spirit of the present invention. In addition, descriptions of well-known functions and constructions are omitted for clarity and conciseness.

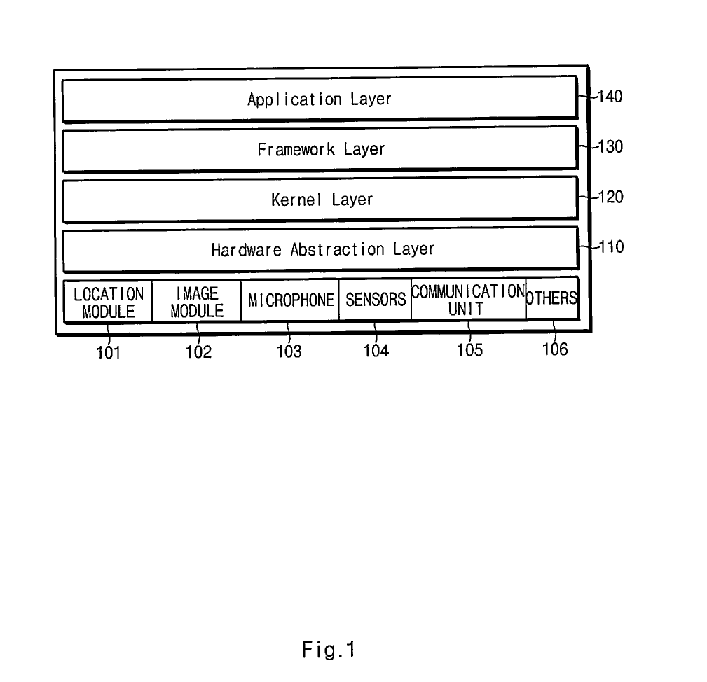

FIG. 1 illustrates a device for collecting stress related information according to an embodiment of the present invention.

Referring to FIG. 1, the device includes one or more devices capable of obtaining information that are connected to a Hardware Abstraction Layer (HAL) 110. In the example illustrated in FIG. 1, the device includes a location module 101, an image module 102, a microphone 103, various sensors 104, a communication unit 105, and other stress information collecting devices/modules 106.

The location module 101 collects the current location of the device. For example, the location module 101 may be a Global Positioning Satellite (GPS) module or an equivalent module using a global navigation satellite system, such as GLONASS or Beidou.

The location module 101 may operate by using a communication network, such as Global System for Mobile communications/Code Division Multiple Access (GSM/CDMA), and a cell IDentifier (ID). The cell ID is a unique number that uses to identify a mobile device in a specific cell network. The location module 101 operating in this way may be understood as a GSM/CDMA module or a cellular network module. For example, such a module may fundamentally use a triangulation algorithm.

The location module 101 may also operate by using an Internet Protocol (IP) address or a WiFi or Bluetooth Media Access Control address (MAC).

The location module 101 may use any of the above-described techniques to collect information on the current location or may combine two or more of the above-described techniques to collect information on the current location. In the latter case, the accuracy of location information on the location of the device may be enhanced.

In certain embodiments of the present invention, the specification focuses on a specific location in which a user is interested, not the current location of the device. In this case, the location module 101 may be operated by a user input interface (not shown). That is, the location module 101 described in the detailed specification may generally mean a tangible device/part for performing a specific function, but is not necessarily limited thereto. The location module 101 may be implemented as a software component as well as a hardware component or may be integrated into another hardware component (for example, a user input interface). Such a variation or expansion may be applied to other components to be described below, within a scope obvious to a person skilled in the art.

The location module 101 may autonomously generate location information but may also generate location information in conjunction with an application loaded on the device 100. For example, the location module 101 may obtain the current GPS coordinates of the device. As another example, the location module 101 may obtain the current location information of the device 100 as a type of MAC address.

Herein, familiar geographical location information (for example, address, street, and building information) may be more meaningful than such mechanical/technological location information. For example, when it is determined that a user device is located at a certain place, data that is preliminarily processed based on data stored in a device or a server (for example, map data) may be more useful than raw data that is initially collected. That is, whether corresponding GPS indicates home, the workplace, or a street may be more meaningful than a GPS value of the user device in terms of user stress management. As such, although it may be needed to be associated with an internal or external application/Data Base (DB) in order to produce data for utilizing stress analysis, such processed data may also be understood as data collected by the location module 101.

Other processed data (such as illumination and humidity data) to be described below may also be understood as data collected by a corresponding module.

The image module 102 may be an image sensor, e.g., a front/rear camera located on the device. The image module 102 may collect images captured by the camera and data processed therefrom.

The microphone 103 may be located on the device or may be an extended part that is connected to the device through a jack connecting unit like a headset. The microphone 103 collects voice data and data processed therefrom.

Various sensors 104, e.g., an inertial sensor, a temperature/humidity sensor, an optical proximity sensor (illumination sensor), a dust sensor, an ultraviolet sensor, an infrared sensor, and/or a touch sensor are included. For example, the inertial sensor may include an accelerometer and a gyroscope. The touch sensor may be implemented as an ultra-thin type display panel such as a Thin Film Transistor-Liquid Crystal Display (TFT-LCD), an Active Matrix Organic Light Emitting Diode (AMOLED), or a flexible display. The above-mentioned sensors are exemplary ideas, and sensors applied to the present invention are not limited to the sensors above.

Various pieces of information may be collected through the communication unit 105. For example, user related data stored in a server, traffic congestion information around a user device, and/or information on other devices linked to the user device may be collected. The communication unit 105 may generally be a wireless interface module such as 3^{rd} Generation/4^{th} Generation (3G/4G) communication, Bluetooth, Wi-Fi, or Near Field Communication (NFC) module, in the case of a mobile computing device (such as a smart phone, a tablet, a smart watch, smart glasses, etc.). Further, the communication unit 105 may also be a wired connection interface module that connects a device to another device or connects a device to a Local Area Network (LAN).

The above-described devices/sensors/modules are connected to the HAL 110. The collected information may be delivered, through a kernel layer 120, to an application layer 140 that operates on a framework layer 130. In accordance with an embodiment of the present invention, such a process may be implemented in a manner in which an Android framework and Dalvik virtual machine are executed through a Linux kernel and an application on the virtual machine is executed. However, the processes may also be applied to other Operating Systems (OSs) through appropriate variations. For example, some OSs do not need to execute a virtual machine.

In the embodiments of the present invention described herein, some data is collected and quickly used while other data is collected and used over a relatively long time. For example, when a user enters a place, such as a construction site, where there is a lot of dust, the dust sensor of a user device may sense the concentration of dust in the air and immediately inform the user that there is a need to leave the place or wear a mask, if the sensed data exceeds a certain reference value. Such sensing may occur once or stop when the user (user device) leaves that place. However, the inertial sensor may determine how much the user moves a day, based on information collected from the motion of the user device throughout the day. Such information is collected and stored.

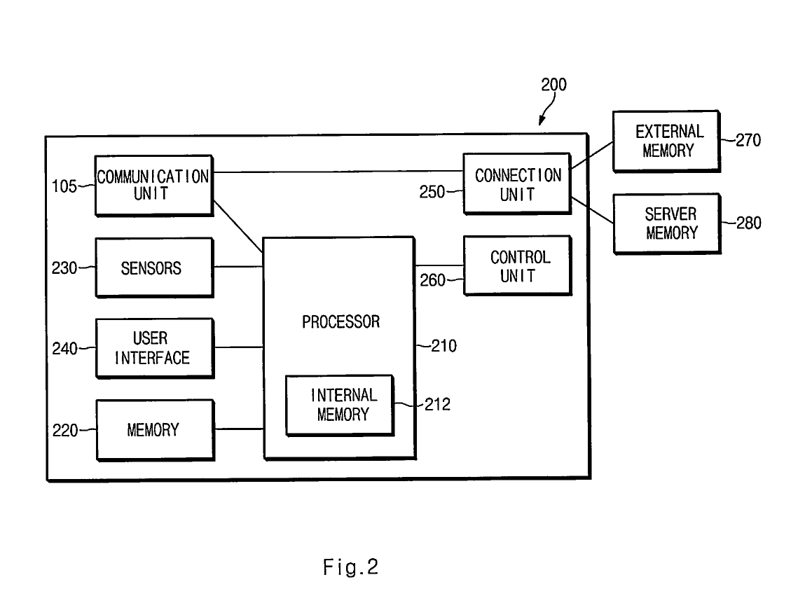

FIG. 2 illustrates a system for storing stress related information according to an embodiment of the present invention.

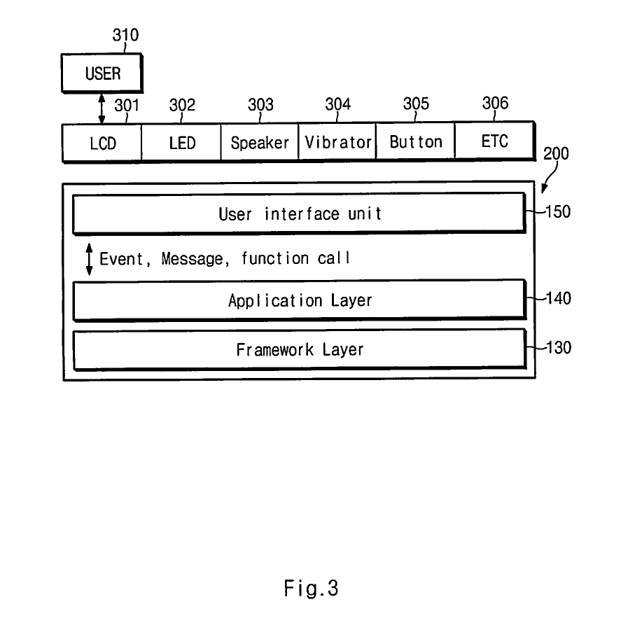

Referring to FIG. 2, the system includes a device 100, an external memory 270, and a server memory 280. The device includes a communication unit 105, a processor 210, a memory 220, sensors 230, a user interface 240, a connection unit 250, and a control unit 260. Some of the devices/modules included in the device 100 illustrated in FIG. 2 are the same as those illustrated in FIG. 1. For example, the sensors 230 in FIG. 2 may be understood as including the location module 101, the image module 102, the microphone 103, and the sensors 104 illustrated in FIG. 1. The user interface unit 240 in FIG. 2 may correspond to the user interface unit described with respect to FIG. 1 and may also correspond to a user interface unit 150 in FIG. 3 as will be described below.

The processor 210 includes an internal memory 212. This internal memory 212 may be a kind of cache memory. The processor 210 is connected to a memory 220. The memory 220 may be a Random Access Memory (RAM) or a storage device such as a Solid State Drive (SSD) or a Hard Disk Drive (HDD).

The processor 210 is connected to the connection unit 250 through the control unit 260, such as a controller, in order to be connected to the external memory 270 or the server memory 280. In order to be connected to the server memory 280, the processor 210 may use the communication unit 105. The external memory 270 may be an extended memory of a mobile computing device, a Universal Serial Bus (USB) memory, an external hard disk, or an optical disk. The server memory 280 may be connected through a wired/wireless network and may be a memory that is included in a server located remotely from a user device. When a stress management process is implemented in a cloud computing system, the memory 220 may be removed from the device 100 and collected data may be stored in the server memory 280. Such a variation may be applied to particular embodiments by a person skilled in the art.

According to the type of the collected data or utilization method of the collected data, the collected data may be temporarily stored in the internal memory 212 or may be stored in the memory 220, the external memory 270, or the server memory 280 for a long time or permanently/semi-permanently. The stored data is analyzed and utilized for user stress management.

A stress analysis result may be displayed to a user.

FIG. 3 illustrates a device for providing stress related expressions according to an embodiment of the present invention.

Referring to FIG. 3, data processed and analyzed by applications executed on an application layer 140 are provided to a user interface unit 150 by using a specific event, message, or function call. A configuration of the user interface unit 150 is described below.

A stress analysis result of collected data may be displayed on a display panel such as an LCD 301. The display panel is not limited to the LCD but may also include an LED based display, a flexible display, and/or an AMOLED display.

The analysis result may also be expressed with an LED 302. It is possible to display a state by merely emitting light from the LED 302, or through flickering or color changing of the LED 302. For example, the LED may be set so that it performs green, yellow, or red flickering according to different user stress states.

The analysis result may also be output through a speaker 303. For example, a voice pattern according to the analysis result may be provided to a user through the speaker 303. Alternately, a certain alarm may be provided. For example, when it is determined that a user 310 is in an extremely stressed state, calm classical music or a calming sound from nature may be provided through the speaker 303.

An analysis result may also be delivered to a user through the vibration of the device 200. The vibration may be generated by a vibrator 304 that is located in the device 200. The vibration may be generated in various predefined patterns besides periodic vibration that is generated for a certain time at certain intervals. Such a vibration pattern may also be defined by a user.

A user may interact with another user through a button 305 with respect to the analysis result. The button 305 may also be a physical button installed on the device 200 or a software button. The physical button may operate by applied pressure. The software button may operate by sensing static generated by a user's touch, by sensing touch pressure or by combining two of the above methods. When operating by sensing the touch pressure, it is possible to operate according to the presence or absence of pressure, or it is possible to operate differently according to the degree of pressure.

When the analysis result is provided, a button display (or button option) may also be provided in addition to the above-described methods. In this case, a user 310 may provide appropriate feedback on a stress analysis result through the button 305. For example, a device may collect a quantity of motion generated for daily work, and if the quantity of motion exceeds a certain reference value the device may provide, to the user 310, a guide to daily work. An alarm is provided along with a button for verification or evaluation, and the user 310 may make a simple selection (for example, “It was a tough day”/“It was an ordinary day”) for the alarm through the button or provide an evaluation (for example, “The stress level of daily work—High/Middle/Low”) on daily work.

In another example, for an arm band or a smart watch device, a selection button or an option for button selection may be provided along with a change in the operation state of the LED 302 attached to the device (for example, flickering or changing color). The user 310 may respond to a stress situation by selecting a provided selection button or a button located on the device 200.

The device 200 may interact with the user 310 using various user interface units 150. There may be various examples in addition to the above-described exemplary interfaces. Also, multiple interface units may operate together. For example, while the device 200 vibrates, the LED 302 may perform a flickering operation and a certain button 305 may be displayed on the LCD display 301 of the device 200.

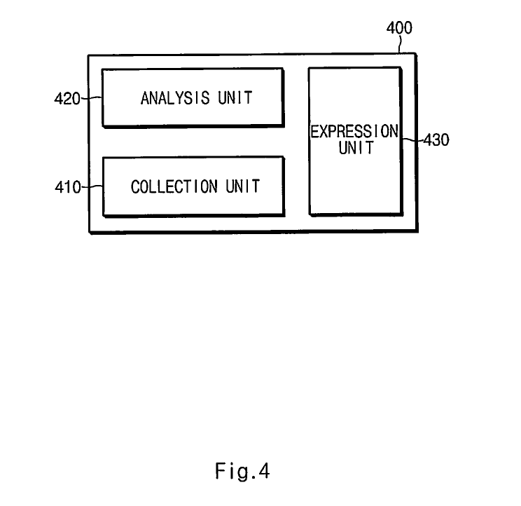

FIG. 4 illustrates a device providing stress management according to an embodiment of the present invention.

Referring to FIG. 4, a stress managing device 400 includes a collection unit 410, an analysis unit 420, and an expression unit 430. The collection unit 410, the analysis unit 420, and the expression unit 430 may correspond appropriately to the sensors 230, the processor 210, and the user interface 240 of the above-described device 100, respectively. For example, the collection unit 410 may include a location module 101 and an image module 102. The analysis unit 420 may be a processor 210 or a processor of a server that is located remotely from a device. The expression unit 430 may correspond to an appropriate exemplary component of the user interface units 150.

In addition to the components illustrated in FIG. 4, the stress managing device 400 may further include appropriate components according to other embodiments or variations. For example, the device 400 may include a storage unit for storing collected information and/or a communication unit for collecting data from an external server.

The collection unit 410 may collect all kinds of information related to stress. The information collected by the collection unit 410 may be classified as follows.

| | |
|---|---|
| (1) Sensed data | (2) User data |
| (3) Internal data | (4) External data |

Such data classification criteria are arbitrary, and some data may be included in two or more groups. Some other forms of data may be inappropriately classified when classified as above. However, such classification is provided for descriptive convenience and is not intended to limit the details or type of data to be collected in the present specification.

### (1) Sensed Data

The collection unit 410 may collect information from various sensors of a device in order to sense a stress state. For example, the collection unit 410 may use an inertial sensor to sense the motion of a device. Also, such a device's motion may be continuously collected for a predefined time or without a predefined time constraint. For example, a quantity of all motions of the device may be collected for a day (24 hours). The quantity of all motions collected in this way may be enumerated at specific time intervals and thus used to determine motions by time intervals. The information may be used to determine whether a user has made motions exceeding a reference value for a day, has made motions that depart from an ordinary pattern, or whether a quantity of physical motions exceeds a typical life pattern. Also, when the quantity of motions is less than an average value for previously collected user data or a creation reference value, such information may be used to predict a change in a predictable ordinary action pattern and request verification from a user.

The collection unit 410 may use an inertial sensor to sense a user walking state. The device 400 may use a Pedestrian Dead Reckoning (PDR) technology to record a user walking state. When a user limps on one leg or maintains a walking state in which the left and right sides of the pelvis are not maintained in balance, this may lead to a musculoskeletal disease such as a joint or vertebra disease. The collection unit 410 may continuously sense the user walking state and provide sensed data to the analysis unit 420 to perform analysis.

The collection unit 410 may use a temperature sensor, a humidity sensor, an ultraviolet sensor, etc., to collect information on the environment around the device 400. When the surrounding environment of the device 400 is too cold, too hot, too humid, or too dry or has an overly high ultraviolet index, a discomfort index felt by the user may be affected. Thus, such surrounding environmental information may be collected as information for stress management. The collection unit 410 may use an illumination sensor to collect surrounding environmental information. For example, when day time illumination is too low or a night time illumination is too high, user stress may result. Alternatively, whether there is light in a specific time interval or whether activities have been performed in a day time may be used as a stress element.

The collection unit 410 may use the dust sensor to sense surrounding air components (for example, Volatile Organic Compounds (VOC), carbon dioxide (CO2), etc.) and may use the sensed data as information for determining stress.

The collection unit 410 may use the image sensor to collect information related to stress. For example, information on an eyeball state captured by using a camera of the device 400 may be used to estimate health conditions. If an eyeball is bloodshot and the distribution of a red image exceeds a reference value, it may be determined that a user is under stress.

As another example, it is possible to capture an image showing the appearance of a user by using a camera and to use captured information for determining stress. For example, if as a result of comparing the state of the user appearance captured one week ago with the state of the currently captured user appearance, the device 400 may determine that user weight has decreased sharply, and thus the user is currently under stress.

Dark circles under a user's eyes found in a captured user image or a user skin state grasped through a captured user image may also be collected as information for determining stress.

The collection unit 410 may use a voice sensor to collect information related to stress. For example, it is possible to collect surrounding noise information by using a microphone of the device 400. When noise equal to or higher than specific decibel (dB) level lasts for a specific period or a conversion value for noise generated around the device 400 for a day exceeds a certain reference value, it may be determined that a user is under stress.

As another example, the voice sensor may analyze user voice. The user voice may be collected when a user is on the phone with another person.

Alternatively, the user voice may also be collected when a user inputs a voice command to the device 400 by using the voice sensor. It is possible to collect information on a user tension state through the volume or waveform of the collected voice.

Alternatively, it is possible to collect, by using a voice recognition technology, information on words that a user mainly uses, and to use the collected information as information for managing stress. For example, if a user speaks on the phone more loudly than usual or uses a lot of slang or a lot of apologetic expressions, such information may be used as a basis for determining that the user is under stress.

The collection unit 410 may collect information on the strength with which the user operates the device 400. For example, the display of the collection unit 410 may include a touch panel, which may include a resistive sensor.

Alternatively, there may be a pressure sensor at an appropriate location on the device.

When a user input is performed through a touch panel of the device 400, it is possible to collect information on typing strength, input strength, typographical error frequency, or strength with which the device 400 is grasped. If an operation pattern different from a usual pattern, such as essential tremor is sensed, such operation information may be used as stress data. The operation pattern, such as essential tremor, may be determined from sensing a noticeable increase in typographical error frequency (typing corrections through a cancel button) or the shaking of the device 400 itself through the previously described inertial sensor when a user inputs texts.

The collection unit 410 may use a thermographic camera to collect user stress information. For example, it is possible to regularly or irregularly capture thermographics of the face of a user who uses the device. Captured data may be stored in a DB. Collected thermographic information may be used to provide a notice to a user, if a change equal to or greater than a certain level is sensed, as a result of comparing user thermographics with a reference value, or if thermographics representing that a user is under high stress are sensed. Also, it may also be used for a regular report on a thermographic change.

### (2) User Data

The user data generally includes user-input data and user-related data. For example, user name, age, sex, workplace, address, and other data that are input directly by a user may correspond to the user data. Even if not directly inputted by the user, user-related data may correspond to the user data. Further, the data collected or sensed by a device, but related to the user of the device, may be classified as the user data. For example, data captured by using the camera of the device may or may not be related to a user. However, the sleeping state of the user captured by using the camera is related to the user and thus may be classified as the user data.

The collection unit 410 may collect a time when a specific event of the device 400 occurs and the temporal duration of that event as stress information. For example, a user may completely stop using the device 400 at 11 PM. Since 11 PM, no user input has been provided to the device 400. If the user first manipulates the device (for example, unlocks the device 400) at 6:30 AM the next day, the collection unit 410 may provide, to the analysis unit 420, information that the user has not manipulated the device 400 for seven and half hours, based on such records (for example, a log record). Such information may be used as information on the amount of sleep or rest of a user. That is, the collection unit 410 may collect a sleep start time, a wake-up time, and total sleep hours as stress information.

As another example, an alarm preset on the device 400 may operate at 6 AM. If the time that a user manipulates the device 400 and completely turns off the alarm is 6:40 AM, it may be considered that the user has undergone a state of incomplete sleep for forty minutes and is in a fatigued state that takes forty minutes to turn off the alarm. Thus, the collection unit 410 may collect the time taken to turn off the alarm as stress information.

Information on a user sleep state may be collected by positioning a user device by using a stand so that image capturing may be performed. In this state, a camera captures images of a user sleeping. In this case, it is possible to determine how often the user tosses and turns while sleeping, by analyzing variations in captured images. As an example, a microphone is activated at a certain period while the user sleeps (since a user input is not provided to the device any more) and recording may thus be performed for a certain time.

Alternatively, the collection unit 410 may record tossing and turning sounds, snoring, sleep-talking, etc., and obtain information on a sleep state when noise levels equal to or greater than a predefined reference value (for example, noise generated when the user tosses and turns while sleeping) is generated (on the condition that the microphone is connected to a sensor hub).

Information on the sleep state may also be collected by determining how often the user moves while sleeping, by using a health band that the user wears. The device (for example, a smart phone) according to an embodiment of the present invention may measure the motion of a sleeping user by using the health band (namely, receive and analyze information collected from the health band) and collect information on a user's sleep state.

As another example, a Closed Circuit TeleVision (CCTV) may be installed at a user's home or in the bedroom for the purpose of crime prevention. The CCTV may be one provided as an additional function of a home phone or may be one installed arbitrarily by the user. Alternatively, a tablet including an image capturing function, a notebook including a web cam, a smart camera (for example, Samsung Galaxy digital camera), etc., may correspond to the CCTV.

A device according to an embodiment of the present invention may receive data that may determine the motion of a sleeping user through a local network connection or wireless network connection, from such a CCTV or a device having a CCTV function. As such, the received user motion information may be used for determining stress.

The collection unit 410 may collect information on elements that disturb sleeping while a user sleeps (when user inputs stop or in a predefined night time). For example, it is possible to collect information on noise, calls, text messages, e-mails, or Social Networking Service (SNS) log.

The collection unit 410 may collect, as stress information, user movement information obtained by using a location module. For example, if it takes one and half hours for a user to move from their home to their workplace, when it normally takes one hour, this may cause stress for the user.

As another example, when the user leaves home at 8:15 AM, but normally leaves at 8 AM, this means that the user is more likely to be late for the workplace and it may thus cause stress for the user.

As another example, when it normally takes twenty five minutes for the user to move from home to a place at which the user catches a transfer, the user waits for about five minutes at the transfer place, so that it takes thirty minutes for the user to move from the transfer place to the workplace. As a result, it usually takes one hour for the user to go to work. In this case, if there is no movement from the transfer place for fifteen minutes, it may be understood that transfer is delayed for some reason. This may cause stress for the user. That is, information on such time for moving between places, waiting times at specific places or the time when a user starts moving from a specific place (or when the user arrives at the specific place) may be collected and utilized as stress information.

The collection unit 410 may use user information and the location module to build an average DB on moving time between specific places and may monitor deviations to utilize them as stress information.

### (3) Internal Data

Internal data includes data that is stored in a memory of the device 400. If data is received from a data storage located on the outside of the device 400, although the data is described in the internal data section, it may be classified as external data. For example, data, such as that in the form of an address book or messages, is stored in an internal storage space of a user terminal; but at the same time, all data is stored in a remote server in a cloud computing system environment. Thus, data such as that in the form of an address book/messages may also be understood as external data.

The collection unit 410 may collect, as stress information, information on the number of unread mails, the number of spam mails, the number of business mails, or combinations thereof. For example, when there are many unread business mails exceeding a certain reference value in a user account, it may cause a user stress

As another example, the collection unit 410 may collect, as stress information, details recorded in a user scheduling program (for example, a calendar application). For example, when there are too many schedules, two different schedules overlap, or schedules suddenly change (within a certain time from a certain start time), stress may be caused and such information may be collected as stress information.

The collection unit 410 may also collect financial information such as a user's financial statements, bank balance, holding stocks, recently purchased/sold stocks, E-commerce related information, credit card usage information, etc. The finance related information may cause a sharp change in a user stress state. Accordingly, factors that have a relatively greater impact than other elements on the user stress state may be given more weight. The weight is described below in more detail.

The collection unit 410 may collect contact list information, e.g., information including a total number of people listed in the contact list and the number of people belonging to each group in the contact list. Also, the collection unit 410 may use communication information such as calls, messages, SNS applications, or e-mails of the device 400 to collect information including the total numbers of received/transmitted calls/messages, the number of received/transmitted calls/messages by group, the number of times that a specific word is used, the number of times that specific content is posted, or preference expressions (“like”, “recommend”, “sympathize”, “following”, “share”, etc.) for a certain period.

If a user is an elementary-school student, a middle-school student, a high-school student, a university student, a test-taker, a university student, or a person who looks for a job, a result of a related test (for example, a regular test, an irregular test, or an interview) may be utilized as stress information. Alternatively, it is possible to collect information on a specific period (for example, a test period) related to a school schedule in linkage with the previously described scheduling program and utilize the collected information as stress information.

### (4) External Data

External data includes data that is collected from the outside of the device 400. Typically, location information on the device 400 is collected from a GPS server or a communication company server. The collection unit 410 may use a location module to collect location information on the device 400 from an external server. The device 400 may further include a communication unit to collect and obtain external data. In some embodiments, a specific module of the collection unit 410 for receiving external data may also function as a communication unit.

The collection unit 410 may obtain information on traffic, crime regions, disaster regions, accident regions, congestion regions, etc. Also, it is possible to obtain information on weather from a weather center server. It is also possible to obtain news or entertainment related information from a server of the press or a service provider server. For example, rainy weather may increase user stress. An increase in political news or information on the activity of a musician preferred by a user may change user stress. Such information may also be utilized as stress information.

The collection unit 410 may collect information through another user device linked to the user device 400. In linkage with an electronic device of a family member or a person closely connected to a user's life, it is possible to collect information on the schedule, stress value, health information, and current state of a corresponding user and utilize the collected information as current user stress information. For example, when the stress value of a wife, an employer, or a teacher is in an extremely high state, this may work as an element that increases the stress of a user such as a husband, employee, or a student.

In some embodiments, the user need not make any direct effort to collect data that is used for managing stress. That is, data may be collected while the user is unaware of the collection. This has various advantages as compared to a typical technology that receives, from users, physical information such as age, height, weight and blood types of users and information on symptoms and performs diagnosis.

For example, many health managing applications require a user to input a lot of information for accurate diagnosis. Various items are enumerated such as a user age, sex and physical condition, as well as past disease history, allergies, family history, whether the user has been in a traffic accident, average amount of movement per week, and average amount of alcohol consumed per week. Some items such as Rh blood type, testing positive/negative for a specific antibody, lastly prescribed medicines and when they were prescribed are difficult for a user to know or memorize. Such complexity may make an application useless even though the application may include various medical routines.

In some embodiments, data used for managing user stress is automatically collected. When a user captures self images using a camera, the collection unit 410 may allow a camera module to automatically obtain information on eyeballs or facial skin (even if the user does not separately enlarge the eyeballs when capturing images). Information on the surrounding environment such as noise, temperature, and humidity may also be automatically collected. Further, by using an inertial sensor, if a user simply carries the device 400, it is possible to collect the amount of travel time, the user location, the quantity of user movement, and walking state. When a user reads Internet articles by using the device 400, the collection unit 410 parses whether corresponding articles are related to crime, politics, sports, or finance or are positive or negative, and collects information that may affect user stress. When the user is on the phone, uses an SNS or changes his/her schedule, appropriate data is collected. There is no need for a user to define their stress state and input the state to a device.

A process for increasing the reliability of collected information may also be performed. For example, when the device 400 is in a pocket or bag, it may have very low illumination in a day time, and in this case, a surrounding environment sensor may not operate. A user may keep the device in a specific place to do intense exercise and in this case, the device 400 may calculate an appropriate quantity of motion in addition to the quantity of motion collected by an inertial sensor, based on a user schedule (for example, basketball), an appointment in an SNS, or conversation. By doing so, it is possible to prevent an inappropriate situation from occurring. That is, it is possible to prevent the device 400 from providing, to a user, an exercise recommending message saying that a quality of motion is insufficient although user's strength is actually undermined due to intense exercise.

Pieces of data collected without a user's intentional input are accumulated and analyzed. The pieces of data may be clustered. The collected pieces of data may generate an average value or a meaningful reference value. When data on which a user action pattern in a specific time departs from an average or reference value by a certain level is collected, the device 400 may predict whether user stress increases or decreases and provide corresponding information or expressions to a user.

The analysis unit 420 may use one or more pieces of reference information that may determine stress stored in the device 400. The reference information may be collected and corrected through a network or user input. When a stress state is produced as a numerical value, a finally produced stress value may be obtained based on information collected from one or more devices. Stress reference information stored in the device may divide steps by each element and each step may have a predefined value.

The analysis unit 420 may determine a user stress state based on the collected information. For example, the analysis unit 420 may enumerate elements affecting user stress, calculate the degree of an impact that a user feels on that elements, and represent stress as a numerical value.

For example, weather may have elements such as serenity, cloud, rain, snow, fog, heavy rain, heavy snow, a storm, thunder and lightening, hail, and typhoon, and a user may be under strong stress under the cloud, the heavy snow, the storm, the thunder and lightening, the hail, and the typhoon. Also, the user may not be under stress under weather conditions such as serenity, rain, and fog.

In a special case, the user may also show a tendency that stress decreases under weather conditions such as rain and snow. As such, it is possible to represent, as a numerical value, the degree of an impact that the user feels on each element (for example, weather conditions such as serenity, cloud, rain, and snow) included in a stress causing factor (for example, weather) and determine a stress state.

In accordance with an embodiment of the present invention, factors affecting user stress may be roughly classified into an environmental factor and an individual factor. Such classification is exemplary and various other types of classification are possible. The environmental factor may be a value/information that is determined by a surrounding environment or a society condition irrespective of a user will. For example, weather (rain or snow), main news (incidents or accidents), or a surrounding environment state (temperature/humidity, dust concentration in the air, or noise) may be included in the environmental factor.

The individual factor may include a value/information related to person's details such as person's face, voice, body, personality, habits, social position, economic power, etc. For example, the individual factor may include the state of a face, the state of voice, property, the presence or absence of sleep/rest, the presence or absence (degree) of a free time, whether an appointment has been conducted, or whether a goal has been achieved.

The information collected by the collection unit 410 may be appropriately classified as the environmental factor or the individual factor. For example, surrounding environment information measured by various sensors may be classified as the environmental factor. The sleep state of the user on which images are captured or user data information (the number of unread e-mails/messages) may be classified as the individual factor. Even if values are measured by using the same sensor, one may be classified as the environmental factor and the other may be classified as the individual factor. For example, although surrounding noise measured by using a microphone may be classified as the environmental factor, information on a user voice state on the phone measured by using the same microphone may be classified as the individual factor.

The analysis unit 420 may represent a state index as a numerical value based on information collected by the collection unit 410. For example, when it is assumed that a value corresponding to a weather factor is W, a value corresponding to a voice state is V, a value corresponding to a face state is F, and a value corresponding to an environment state is E, the stress value of user A may be determined using Equation (1).

S(userA)=W+V+F+E (1)

In Equation (1), each element has a value from 1 to 5.

The value W may be collected from weather information that is received from a weather center server based on the location of a user device that is sensed by using a location module. The value V may be voice information collected when a user voice is on the phone. The value F may be collected from information on eyeballs/facial skin when images of a user face are captured by using a camera. The value E may be collected from a temperature, humidity, an illumination, dust concentration, or a noise level that is measured by using temperature, humidity, illumination and dust sensors and a microphone.

Each factor may be determined based on information on one or more elements included in or related to each factor. For example, the value W may be determined based on an amount of cloud W-cloud, a wind velocity W_wind, and a rainfall W-rain, using Equation (2).

W=W_cloud+W_wind+W_rain (2)

Likewise, the value V may be determined by elements such as waveform information on voice included in voice information, a volume of voice, a type of a used word and the value F may be determined by elements such as an eyeball state or a facial state. The factor collected by each module or sensor may be used for producing a stress value by putting together information on the elements included in the factor.

The stress value may be produced in such a way that a weight is provided to each factor/parameter. Such a weight may be provided according to common practice. For example, while clear weather may have no or very little weight, a rainy day may have a high weight. However, applying such a weight is exemplary, the weight may be individually applied or set based on a user input, an analyzed user pattern, or preference. For example, while user A is sensitive to weather but may have a little change in voice according to a feeling (or stress) state, user B is insensitive to the weather but may easily reveal his/her feeling state through voice. In this case, a stress value may be produced by using provided weights as follows.

S(userA)=5.0*W+1.0*V+ . . .

S(userB)=1.5*W+4.5*V+ . . .

The stress (S) of user A and user B may be determined by a weather condition (for example, W=1 under clear weather, and W=5 under heavy snow) and a change in voice (V=1 under calm voice, V=5 under exited voice) by using the equations above. Weights for other factors may also be appropriately reflected to determine stress values. Such weights may be determined by a user input such as a survey.

In an embodiment, a Stress Management System (SMS) of a device may cluster the above-described stress value S into five steps, as follows.

First step: A user is not under stress, but is peaceful (S=0 to 20).

Second step: A user is under low stress which does not disrupt user activities (S—21 to 40).

Third step: A user experiences great tension depending on the situation and feels a little fatigue (S=41 to 60).

Fourth step: A user always experiences fatigue and fails to sleep well (S=61 to 80).

Fifth step: A user fails to eat and insomnia is accompanied (S=a value greater than or equal to 80).

The stress value may be computed as a numeric sum of the environmental factor and the individual factor and the computed value may be classified into several steps, as described above.

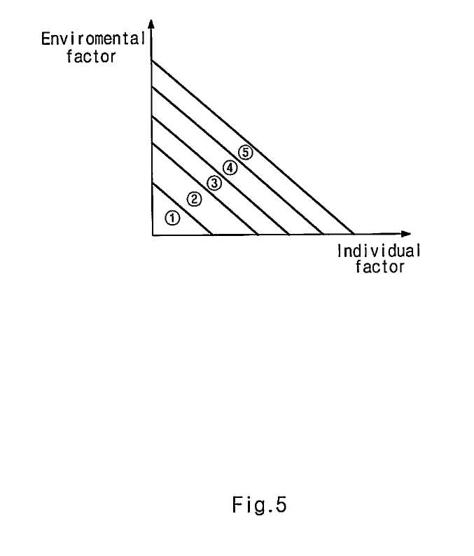

FIG. 5 illustrates a distribution of stress values according to an embodiment of the present invention. In FIG. 5, a number on each area corresponds to each of the above-described steps.

The analysis unit 420 may represent stress as a numerical value based on information collected by the collection unit 410 and record (store) the numerical value. For example, the values of the stress value 22, 25, 28, 35, 27, 18, 17, 20, 25, etc., may be recorded sequentially or at a certain time interval. The values 18 and 17 among the values of the stress value correspond to the first of stress steps defined by the SMS and the others correspond to the second step. In this case, when some stress values are located at the first step, but the values or the duration is less than or equal to a certain ratio, the analysis unit 420 may ignore corresponding data and determine that a user is in the second step, namely, is under low stress that does not disturb user activities.

For example, the values of the stress value 20, 25, 40, 46, 55, 61, 56, 49, etc., may be recorded. In this case, the analysis unit 420 may determine that the stress value is initially located at the second step and then increases sharply within a short time to rise to the third step. In this case, the SMS may determine the current stress state of a user as the third step and provide a notice of the change of a stress state to the expression unit 430 so that an appropriate guide is provided or a user feedback is requested.

As another example, the analysis unit 420 may correct the stress value for incidents and accidents information around a user. In this example, the wording “around the user” may include both a spatial concept and a temporal concept. That is, in addition to information on incidents and accidents that occur near the current location of a user, information on accidents that are significantly spaced geographically from the current location of the user but continuously affect from when the accidents occur (for example, black out in US federal government on Oct. 1, 2013) or accidents that are predicted that they are likely to occur in the near future may also be included in the incidents and accidents information around the user. In this example, a stress value may be corrected as shown in Equation (3).

E′=E−A (3)

In Equation (3), the parameter E′ is a corrected surrounding environmental stress value, the parameter E is a surrounding environmental stress value, and the parameter A is a stress impact index according to incidents and accidents. More particularly, the stress value may be computed using Equation (4).

S=α*P+E (4)

In this example, the parameter S represents a stress value, the parameter α is an arbitrary coefficient, the parameter P represents a user profile value, and the parameter E represents an environment value. In this example, the corrected parameter E′ may be applied in place of the parameter E.

For example, when a user uses a device to read an article on a murder case, user stress may increase if the murder case has occurred within a certain distance from where a user device is currently placed. The device (or the collection unit 410 of the device) may parse article data that the user is currently reading. Parsing may be performed on a keyword basis.

For example, if the collection unit 410 collects the current location of the device 400, recognizes a keyword such as “murder”, “being killed”, or “death” from an article title or details, recognizes, from an corresponding article, a keyword related to a place where an incident has occurred, and provides the recognized place related keyword to the analysis unit 420, the analysis unit 420 may determine a stress value based on the distance between the current location of the device 400 and the place where the incident has occurred.

For an article on an accident, such as a gas leakage or radiation leakage accident, the analysis unit 420 may determine a stress value according to the distance between a leaked place and the current location of the device. In this case, a weight according to the distance may be applied.

Also, individual sensitivity according to an incident type may be applied. For example, when a murder case has occurred but causes a user low stress for several reasons, individual sensitivity may be set as a relatively low value.

As another example, even if the place where the incident has occurred is significantly spaced apart from the current place of the device, the incident may cause a user severe stress. For example, when a sexual assault has occurred over 300 km away from a user, but an acquaintance or family member lives closer to that place, user stress may increase. In this case, the collection unit 410 may collect address information on acquaintance from a user contact list or an SNS, collect incident information from articles or e-mails/messages and use the collected information to produce a stress value. Also, stress correction due to what a criminal lives near a user may be performed in consideration of elements such as whether the criminal lives near a user place (home, workplace, family member's home, etc.), a number of living criminals, and a distance to a criminal place.

In short, a stress impact index A according to an incident/accident may be expressed as shown in Equation (5).

A=(Individual sensitivity)*(Incident and accident type factor)+(User-associated place sensitivity)*(Incident and accident distance factor) (5)

In accordance with an embodiment of the present invention, the analysis unit 420 may extract data that corresponds to a meaningful action pattern from among information on collected user action patterns. To collect the meaningful action data, the analysis unit 420 may have a criterion defining what the meaningful action data is. The criterion may be input from a user or automatically set by using the average of accumulated action data or normal distribution. Alternatively, it is possible to receive and use reference data from an external server.

Herein, meaningful data includes data that excludes data collected for a certain time period from when data measurement starts and data collected for a certain time period from when data measurement ends, in order to ensure stable collection according to a collected information type and increase the reliability of collected data. Also, the meaningful data may include data that is within a preset reference range. However, when it is determined that all measured values are meaningful (for example, measuring a quantity of motion in a daily work or measuring noise), the analysis unit 420 may determine all the values as meaningful data.

When data that is out of a pre-stored range is measured, the analysis unit 420 may request the expression unit 430 to provide feedback to a user, and when a meaningful data range is corrected based on the feedback or the same/similar data is repetitively input, it is possible to automatically ignore that data. That is, the analysis unit 420 stores data and learns based on feedback information to continue to enhance the reliability of an analyzed value.

As an example of extracting data belonging to a meaningful action pattern range, stress according to user touch pressure may be analyzed. When a user performs a touch input on a device where a touch panel display is used as an input unit, it is possible to collect a touch pressure pattern as stress information. In this case, when a touch input starts and ends, very light pressure may be measured. Such a measurement value may decrease an average pressure value for measured all touch inputs and underestimate stress. Thus, the analysis unit 420 may ignore pressure values collected within a predetermined time range from when an input starts and ends.

For example, when a touch input is generally performed within a pressure range of 2 to 10, a normal touch pressure range may be designated as pressure 2 to 10. Such a range may be determined from statistics on collected data. If a value that is out of the range (for example, pressure 15) is measured for a specific time period (for example, three seconds), it is possible to determine that a user is in a stress state. In this case, it is possible to inquire as to whether a user is currently in a stress state, and if the user provides feedback of not being in the stress state, it is possible to increase the upper limit of a normal touch pressure range to pressure 15 (if such feedback is provided several times. If the user provides feedback of being in the stress state, and such a value is measured again, it is possible to determine that the user is in a stress situation.

For example, in a stress situation related to user walking, the collection unit 410 may use an inertial sensor or a location module to measure the movement speed of a user device. Because a user may walk slowly or move through an escalator, a moving walk, or use some other type of transportation, collected movement speed information may vary. In order to measure a meaningful walking speed, it is possible to pre-designate a walking speed range within which human beings typically walk. For example, a speed of 7 km/h may correspond to a typical adult slow jog, and data that exceeds a speed of 7 km/h may be excluded from the analysis, in order to avoid measuring a running state as a walking state. As such, a value representing that a device user is in a walking state is measured.

For example, data corresponding to speeds of 3 km/h to 5 km/h may be measured. If a speed showing a difference equal to or greater than a predetermined reference value from a typical walking speed is measured while measuring the walking speed, the analysis unit 420 may determine that the user is in a stress state.

Alternatively, it is possible to request feedback that verifies whether a user is currently under stress, by using the similar way as that is previously described. An SMS may learn a non-stress situation scope or analyze a stress situation according to user feedback. For example, when it is predicted that a user is late for work or an appointment, the user may quickly walk. In this situation, the user may be under stress, and such stress may be measured by using the above-described way.

The analysis unit 420 may use various analysis techniques. For example, the analysis unit 420 may analyze the average value of collected data, or the average occurrence frequency of events, messages, etc. Such analysis may be performed on specific time, designated periods, or continuous/occasional periods.

For example, it is possible to the average value of noise that occurs from when a user input ends in a night time to when the user input resumes in a morning time the next day. Alternatively, it is possible to analyze the average value of times that are taken to go to work or go home by a user.

The analysis unit 420 may also perform analysis based on a maximum value or a minimum value or based on when a specific value appears very frequently. As such, the average value of specific data may be obtained by using data collected for a specific time period or by using data collected for the entire period. In this example, the wording “the entire period” may be understood as one of since when a device starts collecting data, when the current user of the device registers his/her user account, when collected data is last initialized, or a time that a user determines arbitrarily.

In accordance with an embodiment of the present invention, analysis on a stress value may involve a result of comparison of user data and data on all users using a stress analysis system. That is, collected stress information/numerical values may be stored in a storage device that is located in a network or a server, and may be classified by group (age, sex, job, region, income, etc.)

If it is determined to which group a device user belongs, an amount (numerical value) of stress that a corresponding user experiences is compared with the average value of the same group and a comparison result may be provided to the user device. Consequently, it is possible to compare the level of stress that a specific user experiences with the average level of stress in a group to which the user belongs, and determine whether the former is higher or lower than the latter or corresponds to an average level.

In accordance with an embodiment of the present invention, the collection unit 410 may obtain call related information from a device. The collection unit 410 may obtain Inter Process Communication (IPC) messages. If corresponding messages are classified into an internet call, a voice call, a video call, fax, etc., in class, the numbers of received/transmitted calls of a user device may be obtained and the type of each call may be determined. Further, it is possible to identify when the number of corresponding IPC messages is large and small, and utilize it for stress analysis.

In accordance with an embodiment of the present invention, the collection unit 410 may obtain message/SNS/messenger related information from a device. If obtained IPC messages are classified into an internet message, a voice message, a short text message, an en-mail, fax, etc., in class, the collection unit 410 may obtain the numbers of received/transmitted messages of a user device and determine the type of each message. Further, it is possible to identify when the number of corresponding IPC messages is large and small, and utilize it for stress analysis.

In accordance with an embodiment of the present invention, if an application related to other communication devices is executed, it is possible to grasp the number of communication components by using a destination address or a transmitter address. A stress value may be differently measured according to the number of components.

If a user stress state is determined based on collected data by the analysis unit 420, the expression unit 430 may provide a stress related expression to a user based on this state. The expression unit 430 may deliver stress related information to the user by using various ways, as previously described with reference to FIG. 3.

In accordance with an embodiment of the present invention, the expression unit 430 may use an expression device, such as a display included in a device to provide information on user's current stress state and related content.

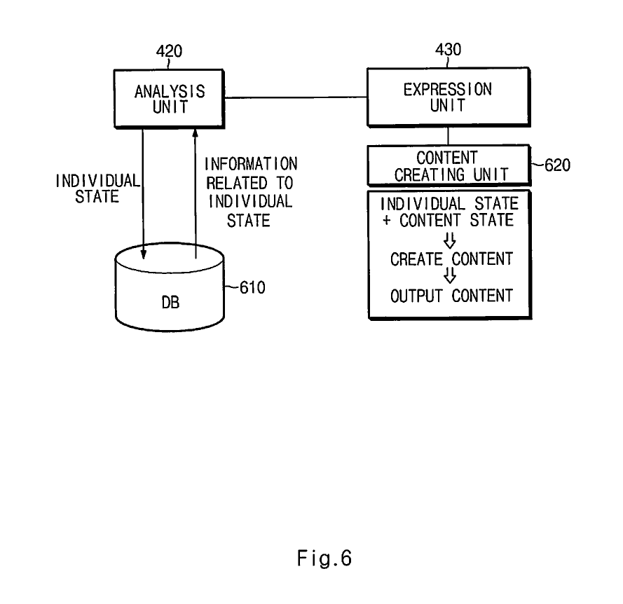

FIG. 6 illustrates an analysis unit and an expression unit of a device for providing stress management according to an embodiment of the present invention.

Referring to FIG. 6, the analysis unit 420 analyzes information collected by the collection unit 410 and provides a result to the expression unit 430. The expression unit 430 provides the provided result to a user through, for example, a display.

The analysis unit 420 may provide information on the user's stress state and related content to the expression unit 430. For example, the analysis unit 420 may analyze a user walking state collected by the collection unit 410 and determine that a user walks more quickly than usual. The analysis unit 420 may then provide information on user's current state (for example, “You walk more quickly than usual”) to the expression unit 430 so that the information is displayed on the display of the device 400. In this case, the analysis unit 420 may provide appropriate content related to the information (for example, “You had better take it easy in this kind of situation”) along with the information. That is, the analysis unit 420 may provide information on a user stress state and related content to an output unit 430.

A content creating unit 620 included in the output unit 430 may put together the information so that a comprehensive result is output to a device user.

The analysis unit 420 may determine a user stress state and share the state with a DB 610. The analysis unit 420 may extract information related to a user state from the DB 610 and determine content to be output so that the content is suitable for the user state. For example, if there is information that dyspnea may be caused if a user heart rate increases, that information may be provided to the output unit 430. The content creating unit 620 may create messages “You are walking more quickly than usual. Decrease your speed or you may experience dyspnea” so that the messages are displayed on the display of the device 400.

The DB 610 may be located in the memory 220 of a device or in a memory 270 or 280 that is located in a remote server.

The expression unit 430 may provide an appropriate expression to a user when one factor to determine a stress value is changed, as in the embodiment above. Also, when a level of stress value calculated comprehensively from several factors is changed (in particular, the level of stress value rises), the expression unit 430 may also provide related information to a user. Also, the expression unit 430 may provide information on one or more stress factors and related content.

For example, when a user schedule is busy in the example above, the content creating unit 620 may provide a guide to a plurality of stress factors such as “Your schedule is busy and you are walking more quickly than usual”. Also, in addition to displaying content created by the content creating unit 620, the expression unit 430 may provide stress related information by using a sound, vibration, an image, a moving picture, etc.

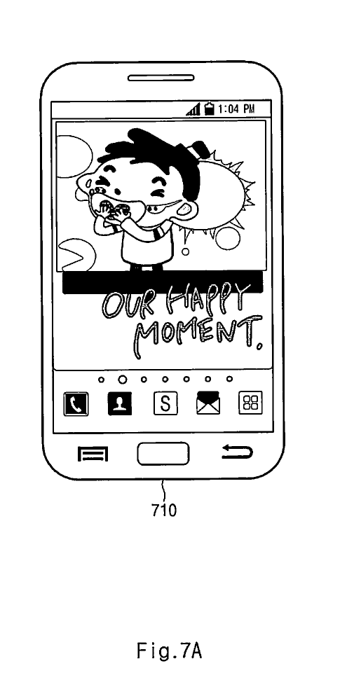

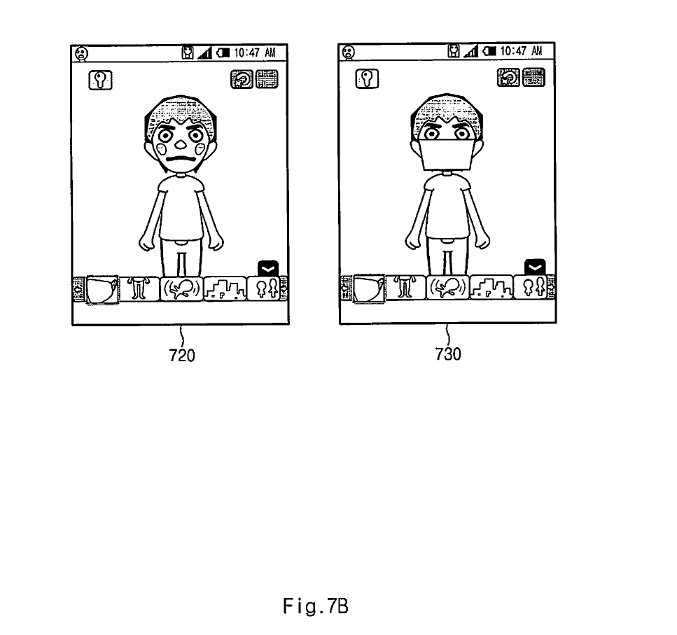

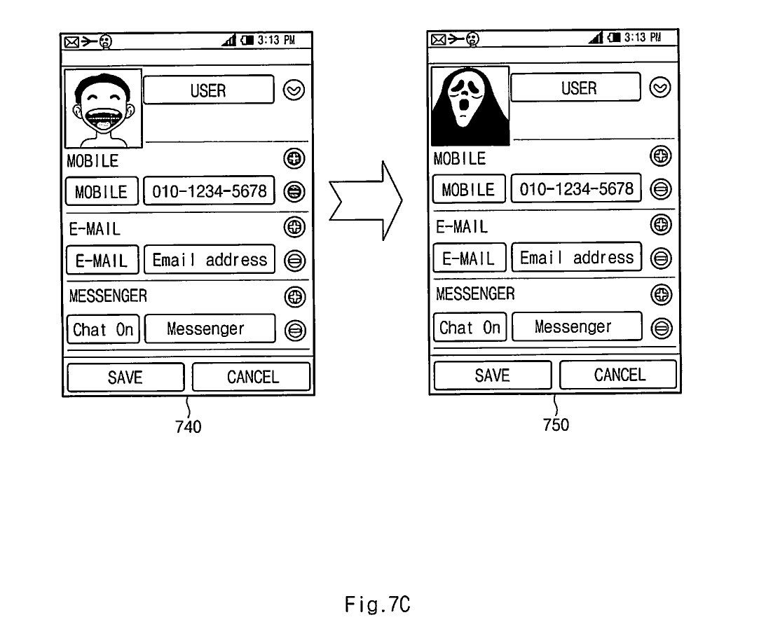

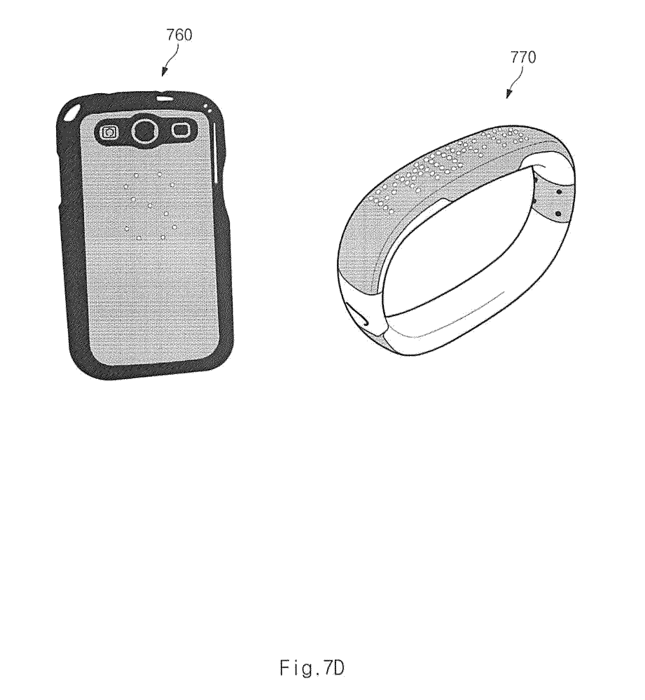

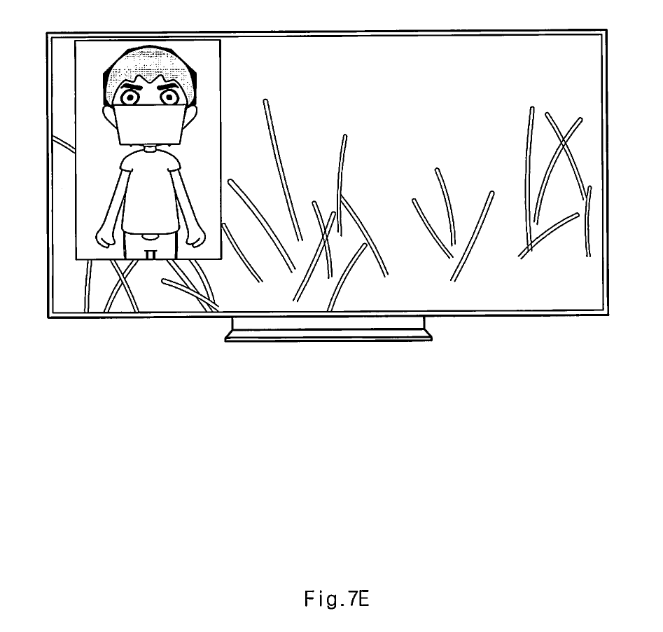

FIGS. 7A to 7E illustrate examples for expressing stress situations according to embodiments of the present invention.

Specifically, FIG. 7A illustrates an example of a stress related expression by using a pop-up window.

Referring to FIG. 7A, if a user who has a respiratory disease such as asthma and is sensitive to dust concentration indoors enters a polluted building, a user stress value may sharply increase by data collected by a dust sensor. In this case, texts, images, moving pictures, etc., may be displayed on a pop-up window on the display of a user device. For example, texts such as “Ventilate the area or you may cough violently” may be displayed, an image where there is yellow dust or a person opening a window may be provided, or a moving picture where there is yellow dust or a person coughing may be played. Also, a voice message such as “Ventilate the area” may be provided, e.g., by using a Text-To-Speech (US) technique.

FIG. 7B illustrates a stress related expression that is provided using an avatar.

Referring to FIG. 7B, the expression unit 430 may display the face and operations of a virtual human representing a user characteristic on the screen 720 of a device so that the current stress state of the user is provided. For example, the expression unit 430 may enable user's avatar to wear a mask, cough or frown, in the example above, on screen 730.

FIG. 7C illustrates a stress related expression that is provided using a user contact image.

Referring to FIG. 7C, the expression unit 430 may re-configure a user's contact information or profile by using a registered photograph. For example, by changing a photograph for user's contact information as well as a user photograph registered with an SNS, from the image illustrated in screen 740 to the image illustrated in screen 750, according to a stress state, it is possible to provide a notice of the current stress level of the user, when other users attempt to contact with the user of the device.

FIG. 7D illustrates a stress related expression is not provided through a screen of the device.

Referring to FIG. 7D, a screen of a device may be turned off or in a locked state. Accordingly, an example of the device 760 may include an LED device in the rear surface or the case of the device for indicating a stress related expression.

In another example of the device 770, such as an arm band or a smart watch, the expression unit 430 may use the flickering of the LED included in the device or the changing of color to provide stress information. For example, a red color may represent anxiety and a blue color may represent comfort. Alternatively, it is possible to change a color according to a level of the stress value. Alternatively, it is possible to change the gradation of a color according to the value of a stress value.

FIG. 7E illustrates a stress related expression provided through an external device.

Referring to FIG. 7E, a device may receive the stress states of other device users and display the received stress states on a screen. Specifically, FIG. 7e represents an example of expressing the stress state related to a user of a first device on a second device.

For example, when the second device is a smart TV and the first device is a smart phone, related content may be displayed on the screen of the smart TV if the stress value of a smart phone user sharply increases. In this example, the first device may connected to the second device over a wired or wireless network, and receive a stress state analyzed based on information collected by the second device and content expressed based on the state through the network. The received content is displayed on the screen of the second device. In this case, the same content may be expressed on the screen of the first device, corresponding content may be expressed in other forms or related content may not be expressed.

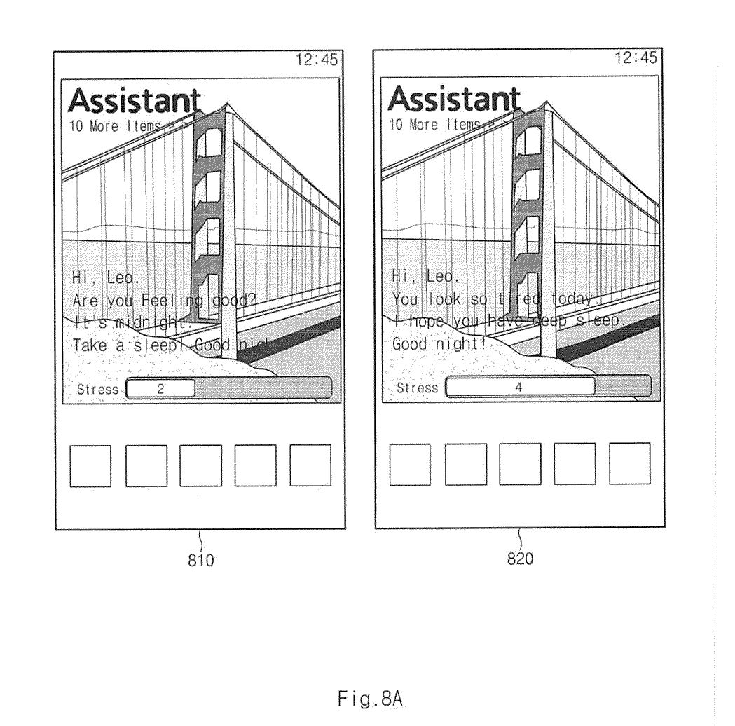

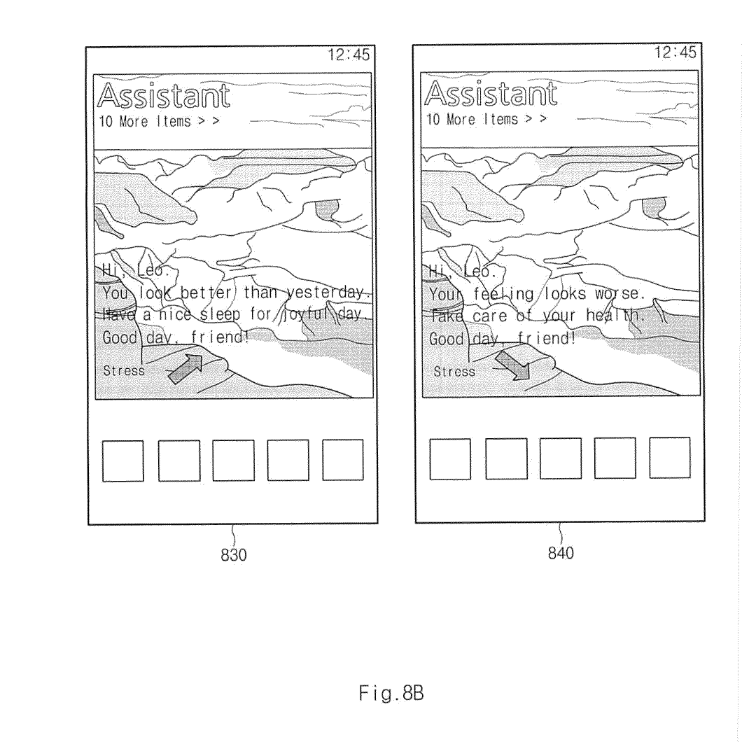

FIGS. 8A and 8B illustrate examples for expressing stress states to a user according to embodiments of the present invention.

Referring to FIGS. 8A and 8B, when a user finishes a daily work or at midnight (0 AM), the expression unit 430 may show day's stress factors and stress change states based on information collected for a day. For example, it is possible to analyze when and where a stress value increases, based on user (device) movement after a user wakes up (operates a device for the first time in the morning). For example, stress may be caused by traffic congestion (receiving real-time traffic information), congested public transportation (delay in transfer time), or weather (rain, snow, strong wind, etc.) Also, when a user schedule is busy (there are a plurality of schedules in a calendar application), there is a lot of work (the number of received e-mails increases), or a user works overtime (the time point that a device starts moving along an after-work route), corresponding information may be collected and it is possible to determine that the user is under stress.

In accordance with an embodiment of the present invention, the expression unit 430 may summarize stress factors before a user goes to bed (in an appropriate time at night) and provide them to the user. In summary, an expression “Sir/madam, you have experienced traffic congestion, you have been busy in working, and worked overtime” may be provided.

Alternatively, by putting together stress information, an expression “Sir/madam, was today tough? Have a good night” may also be provided.

As previously described with reference FIG. 4, the collection unit 410 may collect user data, and in this case, a name (for example, Leo) on a user profile may also be applied in place of sir/madam.

As illustrated in FIG. 8A, a message according to a step of a stress value may be provided based on the step. For example, when the averages of a user stress value (recorded maximum stress value) for a day belong to second step (screen 810) or fourth step (screen 820), information on the stress value and messages corresponding to the steps may be provided.

In FIG. 8B, information generated through comparison with previous (for example, yesterday's) information based on user clustering may be provided. For example, after comparing yesterday's stress state information with today's stress state information, content corresponding to when a yesterday's state is similar to today's state (the step of a stress value is the same), when a stress value (or step) is lower than yesterday's value, or when the stress value is higher than yesterday's value may be respectively provided.

The expression unit 430 may provide the above-described expressions and request a user to provide feedback. For example, when a current stress value is higher than a yesterday's analysis result, it is possible to provide an option to select “Yes/No” along with a message “It seems that your condition is not good today. How's your condition?”. If a user provides “Yes” as feedback, it is possible to lower the stress value by one step or drop it to yesterday's stress value. If a user provides “No” as feedback, it is possible to maintain the current stress level because it is determined that the current stress analysis result is reliable.

In accordance with an embodiment of the present invention, a customized service, which is suitable for a determined stress value, may be provided under regular stress management. For example, when a stress value belongs to a first step, the expression unit 430 may provide a message to recommend dance music. The message may include a selection option, and if the option is selected, corresponding music stored in a device may be automatically played or a user may be connected to a website or application that provides music. When the stress value belongs to second to fourth steps, music suitable for each step may be recommended. When the stress value belongs to a fifth step, classical music suitable for controlling emotion may be recommended.

In accordance with an embodiment of the present invention, when the number of events executed or received/transmitted through applications, sensors, communication units or external devices for a specific period exceed a predetermined reference value (or average value), a pre-stored message, moving picture, graph or notice may be provided to a user.

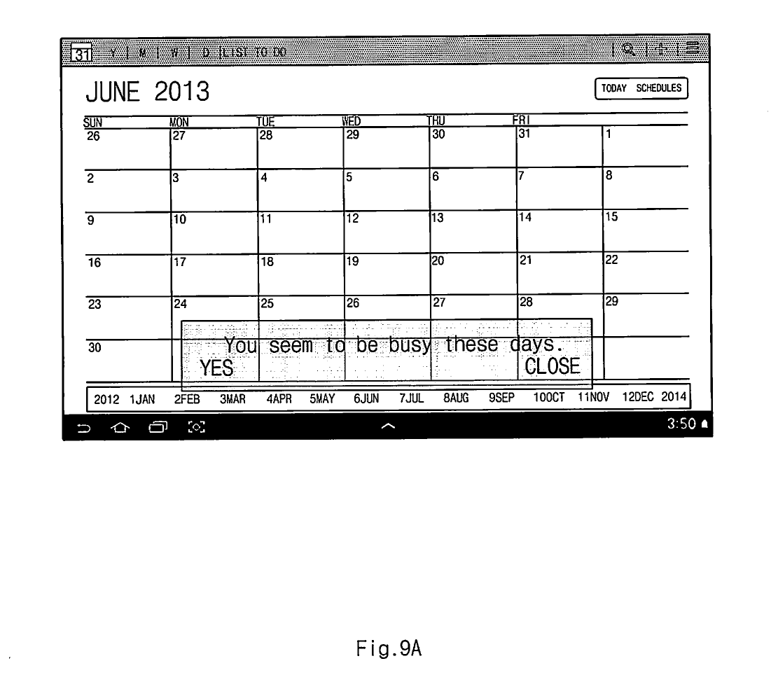

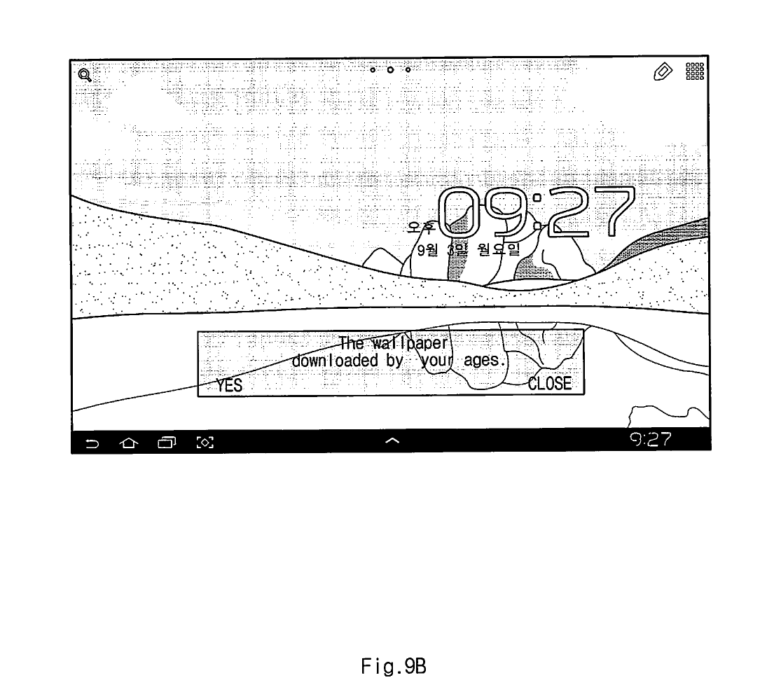

FIGS. 9A and 9B illustrate examples for expressing stress situations according to an embodiment of the present invention.

Referring to FIG. 9A, when the average number of schedules registered per month over the past three months is 15/Month and the number of schedules registered this month (or a specific month) increases by a value greater than or equal to a certain value (for example, three more schedules are added, such that the number of schedules is eighteen), a message “You seem to be very busy these days” may be provide through a display of a device.

Alternatively, while providing the same age's average number of schedules registered per month, after comparing the number of schedules with a DB stored in a server, a message “You seem to be busier than people with the same age.” may be provided.

In a related embodiment, when the number of events executed or received/transmitted through applications, sensors, communication units or external devices for a specific period is smaller or larger than a predetermined reference value, content associated with a corresponding application may be recommended. For example, if the number of calls received for the a current month is a lot smaller than the average number of monthly calls received for the past three months or the same age's average number of received calls stored in an external server (for example, a big data server), the analysis unit 420 may determine that a user is not sociable or a stress factor such as social phobia is increasing.

The expression unit 430 may provide a popup window or message to recommend an SNS related application that may improve sociability. Also, when a user provides feedback such as downloading or pressing cancel button, it is possible to store a corresponding result and determine whether to continue to provide a similar recommendation service in the future.

In a related embodiment, when a user who has normally captures images has not captured a normal amount of images over a specific period, a device transmits the name of an image (or camera) application to an external server and receives content (for example, one of national geographic best images) associated with that application. The expression unit 430 displays the received content to the user and receives feedback from the user. An example of a related expression is illustrated in FIG. 9B.

According to feedback, whether to continue to provide similar images in the future may be determined. For example, if the user shows strong preference for a corresponding image or provides feedback such as recommend or like, the device may provide related images regularly.

In a related embodiment, when the number of times that a specific application has been executed for a specific period is smaller or larger than a predefined reference value, the name of that application may be provided when the user enters a screen on which the application may be used (for example, when a device is unlocked, the main menu of the application is selected or the user enters the home screen).

In a related embodiment, when the number of calls made for the recent one month is too large or small as compared to last month, a pre-stored message “Do you have a new issue?” or “Why don't you make a call?” may be provided if the user unlocks the terminal.

In accordance with an embodiment of the present invention, when a stress value is equal to or higher than a certain level, a stored message, audio data or moving picture may be played, if it is sensed that an illumination in a specific time (for example, at midnight) is in a too low state.

Alternatively, when an illumination in a specific time is very low, an alarm is set, and lighting is dimmed down, stored texts, audio data, and moving pictures may be played. For example, if a user sets an alarm and turns off lighting to sleep, certain words or calm music may be played from a mobile phone.

In accordance with an embodiment of the present invention, the expression unit 430 may provide stress related information to a user in linkage with data received from an external server.

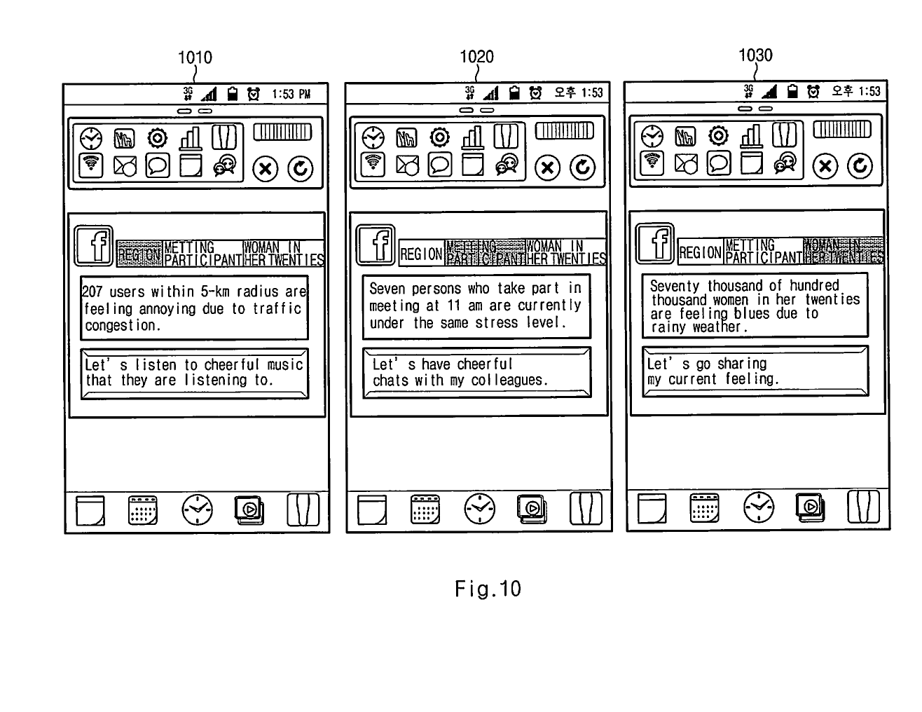

FIG. 10 illustrates a method for providing stress related information in linkage with externally received data according to an embodiment of the present invention.

Referring to FIG. 10, stress information on people located within a certain distance from the current location of a user may be provided to a user on screen 1010. That is, a result that a terminal of another user collects/analyzes stress information associated with that terminal may be again collected as a piece of stress information by a user terminal. Content that people around a user device are equally in an annoyed state may be provided to the user. By this so-called mob psychology, the stress value of a user may decrease. Whether stress reduces may be grasped through user feedback on the provided content.

In a related embodiment, the expression unit 430 provides stress information on users who have registered with schedule management applications to join the same meeting to a device user. An example of a related expression is shown on screen 1020. The expression unit 430 may provide an option (for example, a “Let's have cheerful chats with my colleagues” button) to lower a user stress value. A user may select that option to chat with users to take part in the same meeting, and thus relieve some tension.

In a related embodiment, the expression unit 430 may statistically provide the current stress state for a similar group in conjunction with an external server. An example of a related expression is shown on screen 1030. For example, a device may receive statistics on stress information on women in their twenties among registered users from a server and the received statistics on the screen of a user device.

In accordance with an embodiment of the present invention, in a similar way as described with reference to FIG. 7E, the expression unit 430 may provide a notice of a stress state in linkage with another terminal.

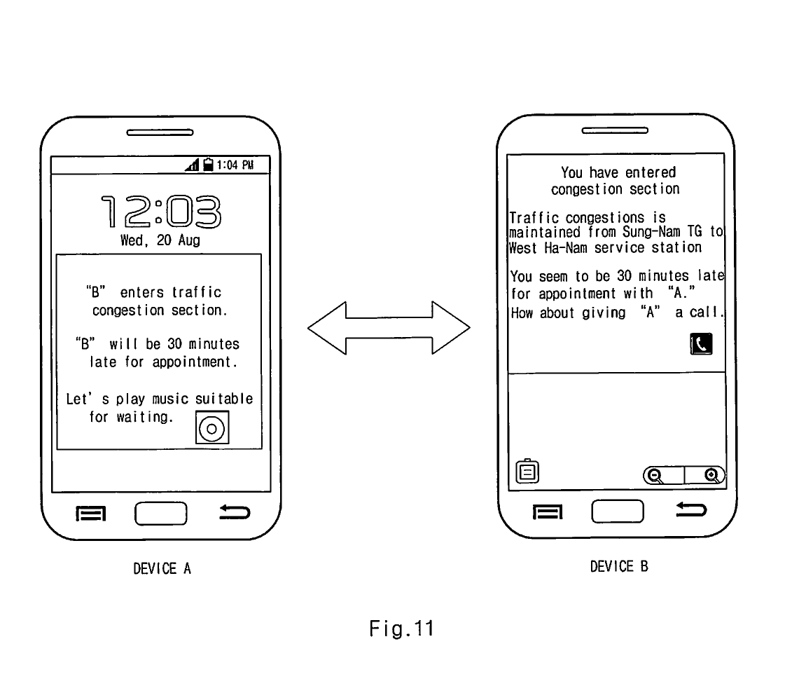

FIG. 11 illustrates a method for a plurality of devices to cooperate and provide stress states to users according to an embodiment of the present invention.

Referring to FIG. 11, both device A and device B may be devices including the collection unit 410, the analysis unit 420, or the expression unit 430 disclosed herein. At least one of device A and device B includes all of three components above.

When a device A's user and a device B's user are supposed to meet on a specific place, the collection unit 410 of device B may use a location module to collect the current location of the device B's user, may collect congestion section information and information on time required for movement from an external server, and may collect information on an appointment time from the scheduling application or memory of device B. The collection unit 410 may also collect (produce) information on an estimated arrival time based on collected information.

The analysis unit 420 of device B produces the stress value of the device B's user based on first information that the estimated arrival time is later than an appointment time, second information on traffic congestion, and information on other environmental/individual factors. The produced stress value may increase as compared to before the first information and the second information are obtained.

The expression unit 430 of device B may provide appropriate content by putting together stress state information on the device B's user and information related to a stress state previously described with respect to FIG. 6.

For example, information on traffic congestion, information that you will be late for an appointment, and a related option (for example, ‘How about giving the terminal A's user a call’) may be provided on the screen of terminal B.

Further, stress information analyzed by device B may be provided to device A through which appropriate information may be provided based on the received stress information on the device B's user.

For example, information that the device B's user enters a traffic congestion section, information that he/she will be late for an appointment, and a related option (for example, “Let's play music suitable for waiting”) may be provided on the screen of device A.

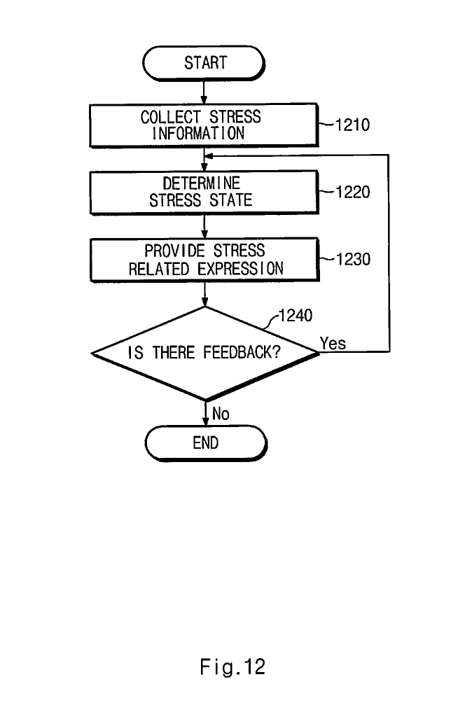

FIG. 12 is a flow chart illustrating a method of managing user stress in a user device according to an embodiment of the present invention.

Referring to FIG. 12, in step 1210, a device collects stress related information. The stress related information may include collected data, user data, internal data, and external data as previously above. In step 1210, a user stress state is analyzed based on the collected information. The device may analyze the user stress state and represent it as a numerical value. The stress state represented as the numerical value may be expressed as a stress value which may be classified into steps.

In step 1230, an expression related to the analyzed stress state is provided. The device may use expression means provided by the device such as texts, images, moving pictures, sounds, vibration or LED to appropriately provide stress related expressions.

In step 1240, the device may receive feedback from a user on the provided expressions.

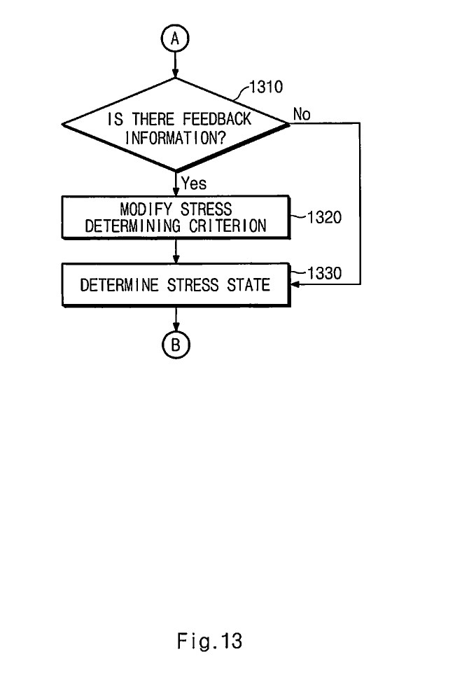

FIG. 13 is a flow chart illustrating a method of managing stress based on feedback received from a user according to an embodiment of the present invention.

Step A in FIG. 13 may correspond to steps 1210 to 1230 in FIG. 12.

Before analyzing a user stress state in step 1330, the device may modify a stress determining criterion in step 1320 based on feedback information collected from a user in step 1310. For example, when stress information that 100 dB noise occurs is collected but feedback that the user is not under stress in the same noise situation has previously been collected from the user (and such feedback has been received several times equal to or more than a certain value), the device may modify the stress determining criterion (for example, lower a weight to be applied when a stress value for the 100 dB noise is calculated)

In step 1330, the device determines a stress state based on the modified criterion and proceeds to step B.

Step B may end the processes or correspond to another appropriate step.

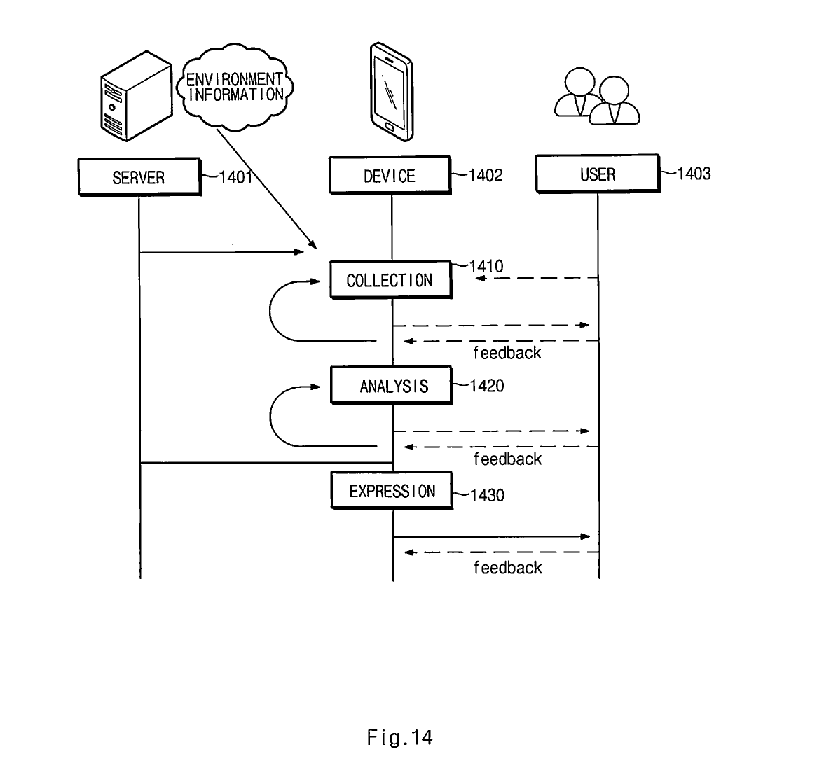

FIG. 14 is a signal flow diagram illustrating a method for managing stress based on feedback received from a user according to an embodiment of the present invention.

Referring to FIG. 14, a device 1402 collects surrounding environment information that may be related to stress. Also, it is possible to collect external data from a server 1401 and collect user data and/or internal data from a user 1403. The device 1402 may provide an appropriate expression 1430 related to stress through information collection 1410 and information analysis 1420. In these processes, feedback on each of the collection, analysis and expression steps may be received from the user.

For example, in information collection step 1410, the device may request feedback on the collected information. According to the feedback received from the user, the device may continue to collect corresponding information or exclude that information from the type of information to be collected in the future.

Based on feedback input from the user in information analysis step 1420 or expression step 1430, it is possible to change an analysis criterion, an expression method or an expression time. For example, when comprehensive stress information on a daily work is provided in a specific time for a day, it is possible to change a time to be provided or it is possible to provide simpler information or more detailed information, according to user feedback.

Stress management using a remote server is also possible.

For example, in FIG. 14, it is possible to transmit collected information related to stress to the server 1401 located remotely from the device. The server 1401 may analyze the stress state of the user 1403 based on the information received from the device, and determine and transmit a related expression to the device 1402 so that the expression is provided to the user 1403. The device 1402 may receive expression data related to stress from the server and provide the expression data to the user 1403. In this case, the server 1401 may include a DB that stores stress information on at least one user group, and the server 1401 may provide, a result of comparing the stress state of the user 1403 with stress information on a user group to which the user 1403 may belong (for example, the average value of a stress value, the stress state, etc.), along with expression data related to stress. The user groups may correspond to groups classified according to one or more of age, sex, region, religion, language, job, income, etc.

The device 1402 may transmit feedback information received from at least one step to the server 1401. The server 1401 may perform stress state analysis based on the feedback or modify a stress state analysis criterion.

Alternatively, the server 1401 may analyze the stress state based on information collected from the device 1402 and transmit the analyzed result (for example, stress value, step, etc.) to the device 1402. The device 1402 may provide content stored in the device to the user as a stress related expression based on the received result.

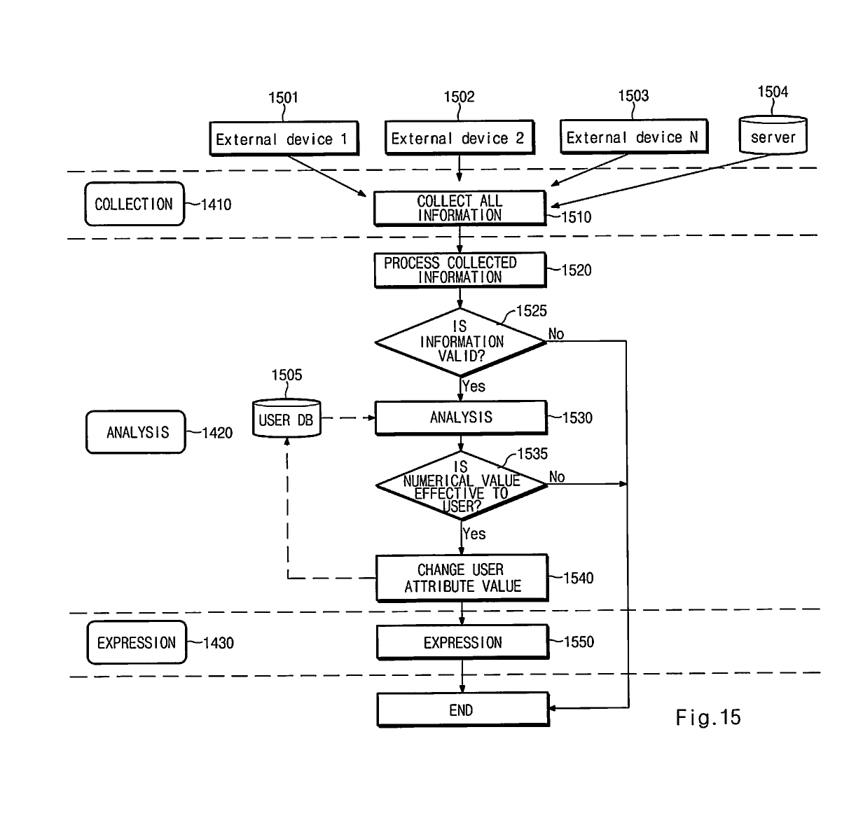

FIG. 15 illustrates a method for managing user stress in a user device according to an embodiment of the present invention.

Referring to FIG. 15, a device 1402 may collect data from a plurality of external devices 1501, 1502, and 1503 and an external server 1504 in step 1510. The collected information may be appropriately processed in step 1520. Processing the collected information is expressed in analysis step 1420 in the sense that a processor takes part in the processing, but it may be considered that such a processing is also included in collection step 1410 as previously described.

In step 1525, the device 1402 determines whether the collected information is meaningful (or valid). For example, when data on a walking state has been collected but the device has moved at a speed of 20 km/h, such data may be excluded.

In step 1530, analysis on meaningful data may be performed. In step 1535, the device 1402 determines whether an analyzed result is meaningful to the user. For example, it is possible to request the user to provide feedback. If the analyzed result is determined as an effective numerical value, user related information stored in a user DB 1505 (for example, user data, a user attribute value, etc.) may be changed based on the numerical value. Data changed in this way may be learned, accumulated, and applied in subsequent analysis step 1530.

In step 1550, a stress related expression is provided to the user.

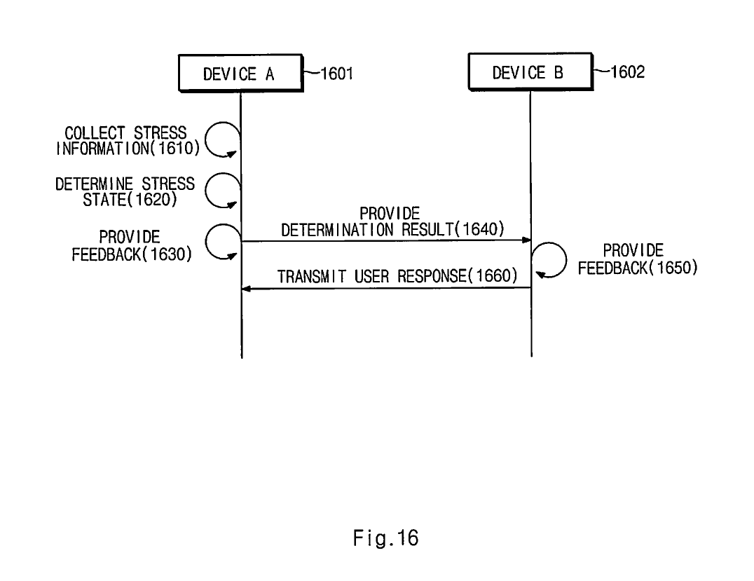

FIG. 16 is a signal flow diagram illustrating a method in which a plurality of devices cooperate and manage user stress according to an embodiment of the present invention.

Referring to FIG. 16, device A 1601 collects stress information related to user A in step 1610.

In step 1620, device A 1601 analyzes a user A's stress state based on the collected information.

In step 1630, device A provides an expression related to an analyzed stress state to user A and receive feedback.

In step 1640, device A 1601 transmits an analyzed stress state to device B 1602. Step 1630 and step 1640 may be performed simultaneously or without order.

In step 1650, device B 1602 provides, to user B, an expression related to a stress state relating to user A and receives feedback from user B.

In step 1660, device B transmits, to device A, a user B's response to the expression provided to user B.

According to the above-described embodiments of the present invention, user stress may be managed continuously/regularly.

According to the above-described embodiments of the present invention, since an SMS accumulates data based on user feedback and learns an analysis criterion, accuracy and reliability of stress management is enhanced.

The component represented as a module or unit for performing a specific function covers any method of performing the specific function and such a component may include a combination of circuit components performing specific functions, or any form of software that is combined with suitable circuits to execute software for performing the specific functions and include firmware, micro-codes, etc.

In the specification, ‘an embodiment’ of the principles of the present invention and the various modified names of such an expression mean that specific characteristics, structures, and properties related to the embodiment are included in at least one embodiment of the principle of the present invention. Thus, the expression “an embodiment” and any other modified examples disclosed throughout the specification do not necessarily indicate the same embodiment.

While the present invention has been particularly shown and described with reference to certain embodiments thereof, it will be understood by those of ordinary skill in the art that various changes in form and details may be made therein without departing from the spirit and scope of the present invention as defined by the following claims and their equivalents.

What is claimed is:

1. A device for managing user stress, the device comprising:
a collection unit configured to collect stress related information;
an analysis unit configured to determine a stress state based on the collected information; and
an expression unit configured to provide a stress related expression to the user, based on the determined stress state,
wherein the collection unit receives feedback on the provided stress related expression from the user, and
wherein the analysis unit uses the feedback to determine the stress state.

2. The device of claim 1, wherein the collection unit collects the stress related information without direct input from the user.

3. The device of claim 1, wherein the analysis unit classifies the collected information into at least one environmental factor and at least one individual factor and converts stress information on each factor to a numerical value.

4. The device of claim 3, wherein the analysis unit produces a stress value, based on a sum of the stress information converted as the numerical value, and generates stress steps based on the stress value.

5. The device of claim 4, wherein the analysis unit corrects the stress value produced by the environmental factor, based on incident or accident information collected by the collection unit.

6. The device of claim 4, wherein the expression unit provides content based on the stress value or one of the stress steps.

7. The device of claim 1, wherein the analysis unit identifies information satisfying a predefined condition among the collected information, and determines the stress state using the identified data.

8. The device of claim 7, wherein the expression unit requests the user to provide feedback on information that does not satisfy the predefined condition, and the analysis unit modifies the predefined condition based on the received feedback.

9. The device of claim 1, wherein the analysis unit compares the stress state of the user with an average stress state of a group to which the user belongs, and provides a comparison result to the expression unit.

10. The device of claim 1, wherein the expression unit provides, to the user, content related to the stress state.

11. The device of claim 1, wherein, based on the stress related information and the stress state of the user, the expression unit provides, to the user, the stress related expression in a specific time, and
wherein the stress related information is collected for a predetermined time period.

12. The device of claim 1, wherein the expression unit compares a first stress state at a first time with a second stress state at a second time and provides the stress related expression related to the comparison.

13. A method of managing stress of a user in a device, the method comprising the steps of:
collecting, by the device, stress related information;
analyzing a stress state of the user, based on the collected information;
providing a stress related expression to the user, based on the analyzed stress state; and
receiving feedback from the user, based on the provided stress related expression,
wherein the feedback is used for analyzing the stress state.

14. The method of claim 13, further comprising receiving feedback on the steps of collecting the stress related information and analyzing the stress state of the-user.

15. The method of claim 14, further comprising changing a stress analysis criterion, based on the feedback on the steps.

16. A method of managing user stress in a device, the method comprising the steps of:
collecting, by the device, stress related information;
transmitting the collected information to a server;
receiving, from the server, an expression related to the stress;
providing the received expression to the user;
receiving feedback from the user, based on the provided expression; and
transmitting the feedback to the server,
wherein the feedback is used by the server for analyzing the stress state of the user.

17. The method of claim 16, wherein the server includes a data base that stores stress information on at least one user group, and
wherein the received expression includes a result of a comparison of the stress state of the user of the device with stress information on a user group including the user.

18. A method of managing user stress, the method comprising the steps of:
collecting, by a first device, stress related information;
analyzing a stress state, based on the collected information; and
transmitting the analyzed stress state to a second device,
wherein the transmitted stress state is used for providing, to the user of the second device, an expression related to the stress of the user of the first device.

19. The method of claim 18, further comprising providing, to the user of the first device, a stress related expression based on the analyzed stress state.

20. The method of claim 18, further comprising receiving feedback from at least one of the users of the first device and the second device,
wherein the feedback is used for determining the stress state.
